(19) United States
(12) Patent Application Publication
(10) Pub. No.: US 2015/0093368 A1
(43) Pub. Date: Apr. 2, 2015

(54) METHODS FOR ENHANCING OXYGENATION OF JEOPARDIZED TISSUE

(71) Applicant: R. Martin Emanuele, San Diego, CA (US)

(72) Inventor: R. Martin Emanuele, San Diego, CA (US)

(73) Assignee: Mast Therapeutics, Inc., San Diego, CA (US)

(21) Appl. No.: 14/553,913

(22) Filed: Nov. 25, 2014

Related U.S. Application Data
(63) Continuation of application No. 13/783,158, filed on Mar. 1, 2013.
Application No. 13/783,158 is a continuation-in-part of application No. PCT/US2011/060747, filed on Nov. 15, 2011.
(60) Provisional application No. 61/413,519, filed on Nov. 15, 2010.

(51) Int. Cl. A61K 47/34; A61K 35/18

(52) U.S. Cl. CPC A61K 47/34; A61K 35/18
USPC 424/93.73

(57) ABSTRACT

The present invention provides methods for preventing the adverse effects of transfusing a patient with blood or blood products compromised by storage lesion. The methods include administering to a patient a pharmaceutical composition comprising an effective amount of a polyoxyethylene/polyoxypropylene copolymer and a pharmaceutically acceptable carrier. The safety and effectiveness of transfusing blood with storage lesion can be increased using the methods of the invention.

### CROSS-REFERENCES TO RELATED APPLICATIONS

This application is a continuation of U.S. application Ser. No. 13/783,158, filed on Mar. 1, 2013, and entitled “Methods for Enhancing Oxygenation of Jeopardized Tissue,” which is a continuation-in-part application of International Application No. PCT/US2011/060747, filed Nov. 15, 2011, which claims priority to U.S. Provisional Application No. 61/413,519, filed Nov. 15, 2010. The subject matter of each of the above-noted applications is incorporated by reference in its entirety.

### BACKGROUND OF THE INVENTION

### I. Tissue Perfusion

It is well known in the art that tissue perfusion is of critical importance during trauma. For example, in 1922, Blalock defined shock as a failure of tissue perfusion. Patients experienced reductions of cardiac output and oxygen consumption during the initial hemodynamic crisis of traumatic and postoperative shock. When continuous monitoring was developed, oxygen consumption was observed to decline prior to the initial hypotensive crisis and was followed by compensatory increases in cardiac output and oxygen consumption. These increases were greater in individuals who survived than in those who died. Similar changes in oxygen consumption were reported by other investigators in patients who developed septic, traumatic, and postoperative shock. Moreover, prospective trials demonstrated improved survival when oxygen consumption was increased by fluids and inotropic therapy. (see, e.g., Shoemaker, W. C., P. L. Appel, and H. B. Kram. 1992. Role of oxygen debt in the development of organ failure sepsis, and death in high-risk surgical patients. Chest 102:208-215)

Reduced oxygen consumption during and immediately after surgical trauma results from inadequate or poorly distributed blood flow and reduced tissue perfusion. This produces an oxygen deficit that can be calculated from measured oxygen consumption minus the oxygen need estimated from the patient's own preoperative values corrected for temperature and anesthesia. Tissue oxygen deficits are greater in patients who subsequently develop multiple organ failure than in patients who recover normally. In lethal cases, oxygen deficits are greater in magnitude and duration than in those who survive multiple organ failure and recover. Moreover, the very early appearance of oxygen debt suggest that reduced tissue oxygenation is the primary event leading to organ failure and death.

In addition, prospective clinical trials have demonstrated that therapy aimed at increasing oxygen consumption in trauma patients decrease mortality, especially when oxygen consumption is maintained at supranormal values. Thus, evidence suggested that reduced tissue oxygenation from maldistributed or inadequate tissue perfusion in the face of increased metabolic need is an early pathogenic mechanism that produces organ failure and death. Possible contributing influences of inadequate perfusion include (a) myocardial and metabolic depression from anesthetic agents; (b) delay or failure to keep up with fluid and blood losses; (c) uneven vasoconstriction by neural mechanisms; (d) preexisting limitations from anemia; (e) chronic cardiac, respiratory, and renal insufficiencies; (f) cytokines, eicosanoids, and other chemical mediators; and (g) inadequate cardiac and respiratory compensatory responsiveness. The first three of these are probably the most important. Data suggest that reduced tissue oxygenation is directly related to subsequent organ failure and death.

Many studies have described complex series of changes leading to and associated with multiple organ failure. The basic question is the identification of underlying pathogenic mechanisms and possible mediators of specific organ system failures so that therapy may be appropriately directed at the primary problem. Many factors affect circulatory function and metabolism, such as age, trauma, sepsis, stress, nutrition, metabolic disorders including diabetes, medications, anesthetic agents, drug abuse, hypovolemia, and other associated illnesses. These and many other influences may limit circulatory compensations. Nevertheless, a common pathway is that the amount of oxygen consumption debt is related to organ failure and outcome. Moreover, oxygen debt is the earliest circulatory event observed with both lethal and nonlethal organ failure.

Much progress has been made in methods and instruments for assessing tissue oxygenation and microvascular function. Oxygen consumption measurements are consistent with tissue oxygen tension measurements using transcutaneous, conjunctival, and subcutaneous oxygen sensors. These studies add evidence supporting tissue hypoxia as the primary underlying physiologic event that produces organ failure and death. Increased cardiac output, oxygen delivery, and oxygen consumption may be physiologic compensations to the underlying tissue hypoxia.

Maintenance of adequate tissue oxygenation is now recognized as important in intensive care units. Venous oximetry obtained by mixing venous oxygen saturation, or central venous oxygen saturation, offers a useful indirect indicator for the adequacy of tissue oxygenation in multiple types of shock (Reinhart, K., and F. Bloos. 2005. The value of venous oximetry. Curr Opin Crit Care 11:259-263). More recent methods for directly monitoring tissue perfusion have been developed (Moore, F. A., T. Nelson, B. A. McKinley, E. E. Moore, A. B. Nathens, P. Rhee, J. C. Puyana, G. J. Beilman, and S. M. Cohn. 2008. Massive transfusion in trauma patients: tissue hemoglobin oxygen saturation predicts poor outcome. J Trauma 64:1010-1023). Near infrared spectroscopy derived tissue hemoglobin oxygen saturation (StO₂) is particularly useful in early prediction of which trauma patients will have poor outcomes. In fact, StO₂ was the only consistent predictor of poor outcome (multiple organ dysfunction syndrome or death) in one large study of such patients. Low StO₂ identified patients who needed massive transfusion, and persistent low StO₂ identified those destined to have poor outcomes. The ultimate goal is to identify these high risk patients as early as possible and to develop new strategies to improve outcome.

### II. Anemia

Anemia can be defined as either a decrease in normal number of red blood cells (RBCs), or less than the normal quantity of hemoglobin in the blood. Anemia produces a decrease in oxygen-carrying capacity of blood. This can be compensated for, but it still decreases reserve and increases the risk of heart attacks and other life threatening complications in affected patients. Anemia due to trauma, hemorrhage or other cause is a common in critically ill patients admitted to intensive care units. The consequences of anemia are compounded in critical illness since the disorders increase metabolic demands (Vincent, J. L., J. F. Baron, K. Reinhart, L. Gattinoni, L. Thijs, A. Webb, A. Meier-Hellmann, G. Nollet, and D. Peres-Bota. 2002. Anemia and blood transfusion in critically ill patients. JAMA 288:1499-1507). Among the many causes of anemia in the critically ill, some of the most important are infection (including sepsis), overt or occult blood loss (including frequent blood sampling), decreased production of endogenous erythropoietin, and immune-associated functional iron deficiency. However, the specific impact of anemia on morbidity and mortality of critically ill patients remains incompletely understood, as is the optimal hemoglobin level for this population. In healthy individuals, for example, only about 25% of the oxygen carried by the blood is extracted during one circuit through the body (normal mixed venous oxygen saturation is around 75%), signifying that there is a significant reserve of oxygen-carrying capacity in the blood. Critically ill anemic patients, however, may have difficulty with hemoglobin levels that would be well tolerated by healthy people as they seem to be unable to utilize the reserve.

To deliver oxygen to the tissues, the RBCs must pass through the microcirculation system where the capillary diameter may vary from 3 to 8 μm. For the 8 μm RBC to navigate these narrow channels, it must retain its deformability. This deformability is dependent on a number of factors including surface area-volume ratio, membrane elasticity, and intracellular viscosity. To maintain these properties, the RBCs depend on the catabolism of glucose and generation of high energy adenosine triphosphate (ATP) via the Embden-Meyerhoff pathway. Loss of their normal biconcave shape and deformability impairs the ability of the RBC to deliver oxygen and remove carbon dioxide from the tissues via the microcirculation system. These senescent RBCs and poorly deformable cells are removed from the circulation as they pass through the splenic circulation (Tinmouth, A., D. Fergusson, I. C. Yee, and P. C. Hebert. 2006. Clinical consequences of red cell storage in the critically ill. Transfusion 46:2014-2027).

Therefore, anemia is not the only cause of insufficient delivery of oxygen to tissues. Diverse severe disorder processes may impair RBC deformability and microcirculatory blood flow and dramatically affect tissue oxygenation. In this setting, transfusion of poorly deformable, 2,3-diphosphoglycerate-depleted stored RBCs with increased vascular adhesion could potentially exacerbate preexisting microcirculatory dysfunction and further impair tissue perfusion. The available evidence suggests that the transfusion of stored RBCs may have adverse effects on micro-circulatory flow and oxygen utilization, particularly in vulnerable patients.

### III. Other Causes of Microvascular Alterations

Microvascular or microcirculatory alterations have been found in many other circumstances (De Backer, D., J. Creteur, M. J. Dubois, Y. Sakr, and J. L. Vincent. 2004. Microvascular alterations in patients with acute severe heart failure and cardiogenic shock. Am Heart J 147:91-99). Microvascular blood flow alterations are frequently observed in patients with heart failure and are more severe in those who do not survive. It has long been known that blood pressure and blood oxygen may be normal in people with early septic shock even though their tissues are poorly perfused. Failure of the microcirculation in these patients is concealed by shunting of blood from arteries to veins without passing through tissues. Increasing the mean arterial pressure from 65 to 85 mmHg with norepinephrine was associated with an increase in cardiac index while microvascular blood flow remained unchanged (Sakr, Y., M. Chierego, M. Piagnerelli, C. Verdant, M. J. Dubois, M. Koch, J. Creteur, A. Gullo, J. L. Vincent, and D. De Backer. 2007. Microvascular response to red blood cell transfusion in patients with severe sepsis. Crit Care Med 35:1639-1644).

Microcirculatory alterations have been observed in association with high risk surgery. In patients submitted to high-risk non-cardiac surgery, Jhanji et al. (Jhanji, S., C. Lee, D. Watson, C. Hinds, and R. M. Pearse. 2009. Microvascular flow and tissue oxygenation after major abdominal surgery: association with postoperative complications. Intensive Care Med 35:671-677) observed that the density and proportion of perfused capillaries was lower in the 14 patients who subsequently developed postoperative complications than in the 11 patients with an uneventful postoperative course. Subcutaneous tissue oxygenation and laser Doppler cutaneous blood flow did not differ between the groups, further highlighting the lack of sensitivity of these methods to detect heterogeneous perfusion. Interestingly, there was no significant difference in global oxygen delivery between the groups. Microcirculatory alterations may also occur in patients undergoing cardiac surgery with or without cardiopulmonary bypass. As in non-cardiac surgery, the severity of perioperative microvascular alterations correlated with peak lactate levels and severity of organ dysfunction after surgery (De Backer, D., G. Ospina-Tascon, D. Salgado, R. Favory, J. Creteur, and J. L. Vincent. 2010. Monitoring the microcirculation in the critically ill patient: current methods and future approaches. Intensive Care Med. 37:1045-46).

Red blood cell rheology may be altered in different disorders, including acute conditions such as patients with sepsis or with inflammatory reactions due to trauma, infection, postoperative states, intra-cerebral hemorrhage, or chronic conditions such as diabetes mellitus or terminal renal failure. Multivariate analysis has demonstrated that the underlying pathology (sepsis, acute inflammatory state, diabetes mellitus, terminal renal failure) is the principal cause of these RBC shape abnormalities (Piagnerelli, M., K. Zouaoui Boudjeltia, D. Brohee, A. Vereerstraeten, P. Piro, J. L. Vincent, and M. Vanhaeverbeek. 2007. Assessment of erythrocyte shape by flow cytometry techniques. J Clin Pathol 60:549-554).

Hemodynamic optimization of these microvascular alterations has been shown to improve outcome in high-risk surgical patients. Although the link between global hemodynamics and microvascular perfusion is quite loose, interventions aimed at improving global hemodynamics also have microvascular effects, which may be mediated by effects independent of changes in global hemodynamics.

In summary, microcirculatory alterations are frequently observed in critically ill patients. These alterations are characterized by a decrease in capillary density and an increase in heterogeneity of perfusion with non-perfused in close vicinity to well-perfused capillaries. Heterogeneous decrease in perfusion is less well tolerated than a homogenously decreased perfusion.

### IV. Assessment of Microvascular Function

Since anemia and the other conditions described above produce inadequate delivery of oxygen to tissues, the patient's tissue oxygenation status should be monitored rather than, or in addition to, hemoglobin when deciding if a transfusion is required during resuscitation. This has customarily been approached by monitoring metabolic markers (base excess/deficit and lactate), which are intermittent measures and thus may not be current with the patient's status, and by invasive monitoring of central venous or mixed venous oxygen saturation.

New technologies, such as direct videomicroscopy or indirect near infrared spectroscopy with a vascular occlusion test, have been developed recently to more directly assess microcirculation in humans. Direct videomicroscopic visualization evaluates the actual state of the microcirculation, whereas the vascular occlusion test evaluates microvascular reserve. The measurement of oxygen tension in skin (TcPO₂) is valuable in assessment of tissue oxygenation, as in peripheral vascular disease, where inadequate blood flow occurs in the legs (Wattel, F., D. Mathieu, and R. Neviere. 1991. Transcutaneous oxygen pressure measurements: A useful technique to appreciate the oxygen delivery to tissues. J Hyperbaric Medicine 6:269-282; Rossi, M., and A. Carpi. 2004. Skin microcirculation in peripheral arterial obliterative disease. Biomed Pharmacother 58:427-431).

Direct microscopic imaging and video microscopy were developed as methods of assessing micro-vascular function in humans (Sakr, Y., M. Chierego, M. Piagnerelli, C. Verdant, M. J. Dubois, M. Koch, J. Creteur, A. Gullo, J. L. Vincent, and D. De Backer. 2007. Microvascular response to red blood cell transfusion in patients with severe sepsis. Crit Care Med 35:1639-1644). A microscope probe is placed under the tongue where blood vessels are close to the surface and micro-vascular activity is captured by video. A computer calculates several parameters of the micro-circulation. Using this technique micro-vascular function was studied in a group of critically ill patients with sepsis. RBC transfusion had no straightforward effect on sublingual micro-vascular flow. There was, however, considerable inter-individual variability. Importantly, there was a dichotomous response, with an improvement in sublingual micro-vascular perfusion in patients with an altered perfusion at baseline and a deterioration in sublingual micro-vascular perfusion in patients with preserved baseline perfusion. Endogenous RBC deformability is thought to be a critical factor in micro-vascular blood flow. Video microscopy has also demonstrated that low-flow conditions such as hemorrhage or cardiogenic shock are associated with a progressive decrease in arteriolar diameter, associated with a substantial decrease in functional capillary density as a result of shutting down some capillaries while others remain perfused with reduced flow (De Backer, D., J. Creteur, J. C. Preiser, M. J. Dubois, and J. L. Vincent. 2002. Microvascular blood flow is altered in patients with sepsis. Am J Respir Crit Care Med 166:98-104). The severity of the decrease in functional capillary density is directly related to a poor outcome. When global flow returns, the microcirculation becomes more heterogeneous as a result of the inflammatory response associated with reperfusion. These alterations were not affected by global hemodynamic variables or the use of vasopressor agents and were totally reversible with the topical application of acetylcholine. It has also been demonstrated that microcirculation improved in survivors of septic shock but failed to do so in patients dying from acute circulatory failure or with multiple organ failure after shock resolution.

Another method of assessing microcirculation is near infrared spectroscopy (NIRS). This measures hemoglobin saturation in muscle 1 cm deep in tissue. There is a significant correlation between StO₂ and oxygen delivery in protocol-driven resuscitation. In an observational trial analyzing 150 patients with trauma during their initial resuscitation, NIRS was found to correlate with the severity of shock, and was found to be more accurate than base deficit in determining severity (Moore, F. A., T. Nelson, B. A. McKinley, E. E. Moore, A. B. Nathens, P. Rhee, J. C. Puyana, G. J. Beilman, and S. M. Cohn. 2008. Massive transfusion in trauma patients: tissue hemoglobin oxygen saturation predicts poor outcome. J Trauma 64:1010-1023). Another recent multicenter trial prospectively collected tissue oxygenation readings in critically injured trauma patients. This study found continuous tissue oxygenation, as measured by NIRS, was as predictive of multiple organ failure and death as base deficit (Kiraly, L. N., S. Underwood, J. A. Differding, and M. A. Schreiber. 2009. Transfusion of aged packed red blood cells results in decreased tissue oxygenation in critically injured trauma patients. J Trauma 67:29-32). The study also showed transfusion of RBCs failed to increase StO₂, confirming the inability of the transfusion to achieve the main purpose of increasing oxygen delivery to tissues. StO₂ has been shown to possess a high negative predictive value in several clinical trials in trauma patients. In patients believed to be at significant risk of shock, those who maintained an StO₂ at 75% or greater in the first hour of arrival in the emergency department had a 91% chance of not developing multiple organ dysfunction, and a 96% chance of survival (Moore, F. A., T. Nelson, B. A. McKinley, E. E. Moore, A. B. Nathens, P. Rhee, J. C. Puyana, G. J. Beilman, and S. M. Cohn. 2008. Massive transfusion in trauma patients: tissue hemoglobin oxygen saturation predicts poor outcome. J Trauma 64:1010-1023).

Diagnostic tools used to assess microcirculation should be able to detect heterogeneity of perfusion. This is best achieved with handheld microvideoscopic techniques. The use of vascular occlusion tests with NIRS investigates microvascular reactivity, another important but different aspect of microvascular function.

### V. Transfusion

Blood transfusion is one of the medical triumphs of the twentieth century. RBC transfusions are a life-saving therapy employed during the care of many critically ill patients to replace losses of blood and to maintain oxygen delivery to vital organs. The goal of transfusions is to increase the hemoglobin concentration, thereby improving oxygen delivery to tissues. RBC transfusions are used commonly in the critical care setting in an attempt to increase oxygen delivery to the tissues and in turn improve tissue oxygenation. The rationale for this therapeutic approach is that an increase in hemoglobin will increase the oxygen carrying capacity of blood and thus provide more oxygen delivery to delivery-dependent tissue (Napolitano, L. M., and H. L. Corwin. 2004. Efficacy of red blood cell transfusion in the critically ill. Crit Care Clin 20:255-268). It has saved many lives of people suffering from acute hemorrhage. Blood product transfusion has also become common during many surgical operations and in persons with anemia or other conditions, with the goal of replacing volume and increasing blood oxygen carrying capacity (O'Keeffe, S. D., D. L. Davenport, D. J. Minion, E. E. Sorial, E. D. Endean, and E. S. Xenos. Blood transfusion is associated with increased morbidity and mortality after lower extremity revascularization. J Vasc Surg 51:616-621, 621 e611-613). The population of patients needing transfusions is steadily advancing in age, and older patients with multiple co-morbid conditions require higher levels of care.

RBC transfusions are commonly used to improve oxygen delivery in acutely ill patients with anemia. However, as discussed above, a number of factors that determine oxygen availability to the cells may not be reliably assessed by hemoglobin levels. In addition, hematocrit is lower in the capillaries than in large arteries and veins as a result of heterogeneous flow distribution, the Fahraeus effect, and interactions between a luminal glycocalyx and plasma macromolecules. Furthermore, the rheologic properties of the transfused RBCs may be altered. In particular, a reduction in RBC deformability can occur during RBC storage or with certain disorders. This may also adversely affect microvascular flow. In a rat model of hemorrhagic shock, the transfusion of stored RBCs did not restore microcirculatory oxygenation in contrast to fresh blood cells. Furthermore, RBC deformability is already altered in sepsis, so the beneficial effects of transfusion of altered RBCs may be even more limited (Piagnerelli, M., K. Zouaoui Boudjeltia, D. Brohee, A. Vereerstraeten, P. Piro, J. L. Vincent, and M. Vanhaeverbeek. 2007. Assessment of erythrocyte shape by flow cytometry techniques. J Clin Pathol 60:549-554).

In many instances where transfusion is used for conditions other than acute blood loss, it is difficult to establish its efficacy. One can calculate the systemic oxygen delivery as proportional to the product of the hemoglobin concentration, oxygen saturation and cardiac output. However, this may not reflect the delivery of oxygen to tissues that need it most. In addition, there are inherent difficulties with tissue specific indicators of cellular respiration and adequacy of oxygen transport and utilization. Simply stated, there is no good way of determining the efficacy of transfusion in all patients. As a consequence, the current criteria for clinical efficacy of transfused blood focus on its physical and biochemical characteristics while having little to do with its function. In clinical practice, physicians rely on hemoglobin concentrations and changes in other crude markers of oxygenation such as mixed venous oxygen and lactate to determine whether a transfusion is efficacious. Unfortunately, recent scientific publications demonstrate that transfused RBCs may be ineffective transporters of oxygen, especially in compromised critically ill patients who have microcirculatory abnormalities (see, e.g., Tinmouth, A., D. Fergusson, I. C. Yee, and P. C. Hebert. 2006. Clinical consequences of red cell storage in the critically ill. Transfusion 46:2014-2027).

A recent study measured StO₂ of trauma patients as they were transfused. Transfusion failed to increase oxygenation in any of the patients. In fact, it caused a decrease in peripheral tissue oxygenation in patients receiving older RBCs. This documents that transfusions are ineffective in improving tissue oxygenation in trauma patients and suggests that stored blood may actually worsen the peripheral vasculature and oxygen delivery (Kiraly, L. N., S. Underwood, J. A. Differding, and M. A. Schreiber. 2009. Transfusion of aged packed red blood cells results in decreased tissue oxygenation in critically injured trauma patients. J Trauma 67:29-32).

It is known that transfusions may be associated with risks. The most immediate danger, hemolytic transfusion reactions, has been largely eliminated by advances in blood typing and matching. Allergic reactions to other components are typically adequately managed with antihistamines and steroids. Dramatic improvements in reduction of transmission of infectious agents have resulted from improved testing and donor selection methods. This has now focused attention on other serious hazards. RBC transfusion may cause adverse effects including the rare, albeit possibly underreported, induction of transfusion-related acute lung injury (TRALI). Like in acute lung injury (ALI) and/or acute respiratory distress syndrome (ARDS), TRALI is thought to result from increased permeability of pulmonary endothelium, edema formation and ventilation to perfusion mismatching with hypoxemia. In TRALI, the increased pulmonary vascular permeability by leukocytes, activated by antibodies or bioactive substances released during storage of RBC units, may be superimposed on a primary ‘hit’ to the pulmonary endothelium. However, the course and characteristics of TRALI, as well as its differentiation from transfusion associated circulatory overload (TACO) remain poorly understood. Pulmonary edema in TACO is thought to be the result of increased hydrostatic pressure due to a hypervolemic state after RBC transfusion. However, there is no sentinel feature that distinguishes TACO from TRALI (Cornet, A. D., E. Zwart, S. D. Kingma, and A. B. Groeneveld. Pulmonary effects of red blood cell transfusion in critically ill, non-bleeding patients. Transfus Med. 2010 Aug. 1; 20(4):221-6).

Therefore, there is an increasing awareness that even when things apparently go well, transfusions may not produce the desired effects and may even cause worsening of disorder or premature death. Worse outcomes in transfused patients have been observed in various settings such as critically ill patients, elderly patients, cardiac surgery/trauma/orthopedic surgical patients, and patients with acute coronary syndrome. In certain studies, patients receiving allogeneic transfusions have had higher mortality rates, higher risk of intensive care unit (ICU) admission, longer hospital and ICU stays, higher postoperative infection rates, higher risk of developing adult respiratory distress syndrome (ARDS), longer time to ambulation, higher incidence of atrial fibrillation, and higher risk of ischemic outcomes compared with non-transfused cohorts (O'Keeffe, S. D., D. L. Davenport, D. J. Minion, E. E. Sorial, E. D. Endean, and E. S. Xenos. Blood transfusion is associated with increased morbidity and mortality after lower extremity revascularization. J Vasc Surg 51:616-621, 621 e611-613). In addition, allogeneic blood transfusions in combat casualties were associated with impaired wound healing, increased perioperative infection rate, and greater resource utilization (Dunne, J. R., J. S. Hawksworth, A. Stojadinovic, F. Gage, D. K. Tadaki, P. W. Perdue, J. Forsberg, T. Davis, J. W. Denobile, T. S. Brown, and E. A. Elster. 2009. Perioperative blood transfusion in combat casualties: a pilot study. J Trauma 66:S150-156).

Blood transfusion is also a strong independent predictor of mortality and hospital length of stay in patients with blunt liver and spleen injuries after controlling for indices of shock and injury severity. Transfusion-associated mortality risk was highest in the subset of patients managed nonoperatively (Robinson, W. P., 3rd, J. Ahn, A. Stiffler, E. J. Rutherford, H. Hurd, B. L. Zarzaur, C. C. Baker, A. A. Meyer, and P. B. Rich. 2005. Blood transfusion is an independent predictor of increased mortality in nonoperatively managed blunt hepatic and splenic injuries. J Trauma 58:437-444; discussion 444-5). In general, more severely ill patients, as measured by either APACHE II (Acute Physiology and Chronic Health Evaluation II) or sepsis-related organ failure assessment (SOFA) score, received more RBC transfusions. Even after correction for baseline hemoglobin level and severity of illness, however, more RBC transfusions were independently associated with worse clinical outcomes (Napolitano, L. M., and H. L. Corwin. 2004. Efficacy of red blood cell transfusion in the critically ill. Crit Care Clin 20:255-268). A randomized controlled trial compared a liberal transfusion strategy (hemoglobin 10 to 12 g/dL with a transfusion trigger of 10 g/dL) to a restrictive transfusion strategy (hemoglobin 7 to 9 g/dL with a transfusion trigger of 7 g/dL). Patients in the liberal transfusion arm received significantly more RBC transfusions. Overall in-hospital mortality was significantly lower in the restrictive strategy group (Napolitano, L. M., and H. L. Corwin. 2004. Efficacy of red blood cell transfusion in the critically ill. Crit Care Clin 20:255-268).

However, transfusion is very common in the treatment of patients with trauma. Typically, transfusion is first used for the replacement of acute blood loss. Later in the course of treatment, patients often receive transfusions for a decreased hematocrit. The intention in this scenario is to increase oxygen-carrying capacity. However, the actual effect of stored RBC transfusion on tissue oxygenation is not well established. Previous studies have been conducted on animal models with mixed results. The strategy of maximizing systemic oxygen delivery through transfusion and other measures in the post injury period has been widely employed. Nevertheless, outcome studies have been disappointing. In fact, multiple retrospective studies show an association between blood transfusion, multiple organ failure, and death (Kiraly, L. N., S. Underwood, J. A. Differding, and M. A. Schreiber. 2009. Transfusion of aged packed red blood cells results in decreased tissue oxygenation in critically injured trauma patients. J Trauma 67:29-32). In patients undergoing surgery for lower extremity revascularization, there is a higher risk of postoperative mortality, pulmonary, and infectious complications after receiving intra-operative blood transfusion. Transfusion in cardiac surgery patients has been associated with increased mortality, higher incidence of postoperative infection, prolonged respiratory support, higher risk of postoperative infection, and higher risk of renal failure. Similarly, in critical care patients, transfusion has been associated with increased overall and ICU 14-day mortality rate, higher 28-day mortality rate, longer length of stay, higher risk of developing ARDS, and higher incidence of bloodstream infections (O'Keeffe, S. D., D. L. Davenport, D. J. Minion, E. E. Sorial, E. D. Endean, and E. S. Xenos. Blood transfusion is associated with increased morbidity and mortality after lower extremity revascularization. J Vasc Surg 51:616-621).

One study used NIRS to demonstrate a significant decrease in the tissue oxygenation in patients receiving packed red blood cells stored for more than three weeks, though a decrease in oxygenation may seem counterintuitive considering the theoretical increase in oxygen-carrying capacity (Kiraly, L. N., S. Underwood, J. A. Differding, and M. A. Schreiber. 2009. Transfusion of aged packed red blood cells results in decreased tissue oxygenation in critically injured trauma patients. J Trauma 67:29-32). Moreover, transfusion of newer blood failed to increase tissue oxygenation. Several potential mechanisms may explain these findings. Recent work has demonstrated significant changes in packed red blood cells after storage. Specifically, S-nitrosohemoglobin concentrations have been noted to decline rapidly after red cell storage. Decreased concentrations restrict the ability to locally control vasodilatation. In the setting of decreased saturation, stored cells would not be able to compensate by increasing flow. Breakdown of red cells results in free hemoglobin. Free hemoglobin scavenges nitric oxide hindering local vasodilatation. This is one of many studies that demonstrate an association between transfusions and diminished organ function and mortality.

The mechanisms by which transfusions produce adverse events are incompletely understood and multi-factorial. The immunosuppressive effects of blood transfusion may be responsible for the observed increase in risk of infection. Blood transfusions have been shown to be independent risk factor for infection. In addition, transfused blood may actually compromise the function of microcirculation in tissues that need it most. Furthermore, allogenic blood transfusion in the first 24 hours after trauma is associated with increased systemic inflammatory response syndrome (SIRS) and death (Dunne, J. R., D. L. Malone, J. K. Tracy, and L. M. Napolitano. 2004. Allogenic blood transfusion in the first 24 hours after trauma is associated with increased systemic inflammatory response syndrome (SIRS) and death. Surg Infect (Larchmt) 5:395-404). Compelling evidence has recently been obtained that transfusion of stored RBCs may have adverse effects on microcirculatory flow and oxygen utilization, particularly in vulnerable patients (Tinmouth, A., D. Fergusson, I. C. Yee, and P. C. Hebert. 2006. Clinical consequences of red cell storage in the critically ill. Transfusion 46:2014-2027). Transfusion of RBCs decreases oxygenation thereby increasing the lung injury score, dose dependently and transiently, in a heterogeneous population of critically ill, non-bleeding patients, independent of prior cardiorespiratory status and RBC storage time (Cornet, A. D., E. Zwart, S. D. Kingma, and A. B. Groeneveld. Pulmonary effects of red blood cell transfusion in critically ill, non-bleeding patients. Transfus Med. 20:221-226, 2010).

In current practice, RBCs can be transfused for up to 42 days after collection. Recent literature has reported that the age of RBCs contributes to complication. A systematic literature review identified 24 studies that evaluated the effect of RBC age on outcomes following transfusion in adult patients. The results are contradictory. Some studies suggest that the age of transfused RBCs may play a role in the morbidity and mortality of adult patients undergoing transfusion, others do not. However, numerous factors can explain these conflicting data (Lelubre, C., M. Piagnerelli, and J. L. Vincent. 2009. Association between duration of storage of transfused red blood cells and morbidity and mortality in adult patients: myth or reality? Transfusion 49:1384-1394). Many of the reports were small, observational cohort, single center studies with heterogeneous populations and variation in the method of reporting RBC age. Notwithstanding, there is considerable evidence that prolonged storage of RBCs can adversely affect clinical outcomes following transfusion. A study in rats reported that transfusion of RBCs after prolonged storage produces harmful effects that are mediated by iron and inflammation (Hod, E. A., N. Zhang, S. A. Sokol, B. S. Wojczyk, R. O. Francis, D. Ansaldi, K. P. Francis, P. Della-Latta, S. Whittier, S. Sheth, J. E. Hendrickson, J. C. Zimring, G. M. Brittenham, and S. L. Spitalnik. Transfusion of red blood cells after prolonged storage produces harmful effects that are mediated by iron and inflammation. Blood. 2010 May 27; 115(21):4284-92). There is also compelling data in people. Patients who developed major infections (n=32, 51%) received more units of RBCs greater than 14 days old (11.7±1 units vs. 8.7±0.7 units, p=0.02) or greater than 21 days old (9.9±1.0 units vs. 6.7±0.8 units, p=0.02), but their total early transfusion requirement was higher than patients without infection (12.8±0.9 vs. 10.4±0.8, p=0.04). In a multivariable analysis controlling for potential confounders, the number of units older than 14 and 21 days remained an independent risk factor for major infections ((Lelubre, C., M. Piagnerelli, and J. L. Vincent. 2009. Association between duration of storage of transfused red blood cells and morbidity and mortality in adult patients: myth or reality? Transfusion 49:1384-1394). Transfusion of blood that is stored for prolonged periods (but still within the currently accepted maximum allowed storage time of 42 days) has been linked to increased risk of complications and reduced survival in patients undergoing cardiac surgery and in other patient populations. A recent study measured StO₂ of trauma patients when they were transfused. The transfusions never increased tissue oxygenation and actually decreased it in patients receiving RBCs older than three weeks. This is highly suggestive that factors in stored blood may influence the peripheral vasculature and oxygen delivery (Kiraly, L. N., S. Underwood, J. A. Differding, and M. A. Schreiber. 2009. Transfusion of aged packed red blood cells results in decreased tissue oxygenation in critically injured trauma patients. J Trauma 67:29-32).

In summary, it has been shown that: (1) RBC transfusion does not improve tissue oxygen consumption consistently in critically ill patients, either globally or at the level of the microcirculation; (2) RBC transfusion is not associated with improvements in clinical outcome in the critically ill and may result in worse outcomes in some patients; (3) specific factors that identify patients who will improve from RBC transfusion are difficult to identify; and (4) lack of efficacy of RBC transfusion is likely to be related to storage time, increased endothelial adherence of stored RBCs, nitric oxide binding by free hemoglobin in stored blood, donor leukocytes, host inflammatory response, and reduced red cell deformability.

Therefore, new technologies are needed to improve the safety and efficacy of RBC transfusions. New technologies are also needed to replace RBC transfusions under conditions where they have been shown to be ineffective or potentially even harmful.

Due to the risks associated with anemia and blood transfusions, alternative treatments of anemia in the critically ill have been explored. Much effort has been expended for over 30 years to develop blood substitutes. The first substitutes tried were perfluorocarbons, chemicals with high oxygen solubility. An emulsion of perfluorocarbons, Fluosol-DA 20%, was extensively studied and was approved in the United States for delivery of oxygen through catheters during angioplasty. However, this emulsion was not approved as a blood substitute because it failed to carry sufficient oxygen (Castro, C. I., and J. C. Briceno. 2010. Perfluorocarbon-based oxygen carriers: review of products and trials. Artif Organs 34:622-634). Many other approaches using perfluorocarbons, modified hemoglobin or other substance have been developed, but none have progressed in clinical trials because of lack of efficacy and/or toxicity (Lowe, K. C. 2001. Substitutes for blood. Expert Opin Pharmacother 2:1057-1059).

Another approach, administration of exogenous human recombinant erythropoietin (epoetin alpha) has been shown to raise reticulocyte counts and hematocrit levels, and to reduce the total number of units of transfused blood required in critically ill patients (Vincent, J. L., J. F. Baron, K. Reinhart, L. Gattinoni, L. Thijs, A. Webb, A. Meier-Hellmann, G. Nollet, and D. Peres-Bota. 2002. Anemia and blood transfusion in critically ill patients. JAMA 288:1499-1507). However, this does not address the need for improved oxygen delivery to tissues during times of crisis. Anemia, disease and storage of blood for transfusion can all alter red blood cells making them less able to deliver oxygen to tissues where it is needed most. Lack of sufficient oxygen then damages tissue further, especially the microvasculature, causing further reduction in oxygenation leading to organ failure and/or death.

Therefore, what is needed is a pharmaceutical composition that can improve delivery of oxygen to tissues through the microvasculature of critically ill patients who have lost flexibility of RBCs; restore the flexibility of rigidified RBCs facilitating their passage through the microvasculature; maintain normal oxygenation of tissue in patients at risk of shock there by preventing development of shock; maintain normal oxygenation of tissue in patients at risk of disorders caused by localized tissue ischemia such as crisis of sickle cell disease and acute limb syndrome of peripheral artery disease thereby preventing development of the disease complication; improve both the safety and efficacy of RBC transfusions; improve the ability of transfused RBCs to deliver oxygen through the microcirculation of vulnerable tissues where it is needed; and counter the deleterious effects of storage lesion on transfused blood.

### SUMMARY OF THE INVENTION

Methods for improving the oxygenation of jeopardized tissues are described herein. The methods are useful for decreasing the need for transfusions, improving the safety and efficacy of blood transfusions, improving organ transplantation and for the treatment of patients suffering from conditions or disorders that affect the oxygenation of blood and tissues. Exemplary conditions or disorders to be treated using the methods described herein, include but are not limited to: anemia, trauma, hypovolemia, inflammation, sepsis, microvascular compromise, sickle cell disease, acute chest syndrome, peripheral artery disease, myocardial infarction, stroke, peripheral vascular disease, macular degeneration, acute respiratory distress syndrome (ARDS), multiple organ failure, ischemia (including critical limb ischemia), hemorrhagic shock, septic shock, acidosis, hypothermia, and anemic decomposition. The methods described herein are also useful for the treatment of patients in need of transfusion, patients undergoing surgery (including plastic surgery), and patients with blood disorders. Furthermore, in one embodiment, the methods described herein are useful for preventing the adverse effects of transfusing a patient with blood that has been compromised by storage lesion. The compositions and methods described herein are also useful for preserving the function of a donor organ.

In one embodiment of the methods provided herein, an effective amount of a pharmaceutical composition containing the polyoxyethylene/polyoxypropylene copolymer described below is administered to a patient.

In accordance with another embodiment, a pharmaceutical composition containing the polyoxyethylene/polyoxypropylene block copolymer described below is combined or admixed with blood or blood products, such as the patient's own blood or the blood of a blood donor and the combination is administered to a patient such as in the form of a blood transfusion. Alternatively, the pharmaceutical composition containing the polyoxyethylene/polyoxypropylene block copolymer described below is administered separately to a patient either prior to, concomitant with, or immediately after a transfusion.

In accordance with another embodiment, a pharmaceutical composition containing the polyoxyethylene/polyoxypropylene block copolymer described below is administered to an organ donor prior to organ donation, an organ to be transplanted into a patient is perfused with the polyoxyethylene/polyoxypropylene block copolymer described below, or the polyoxyethylene/polyoxypropylene block copolymer described below is administered to an organ recipient patient after organ transplantation.

Also provided herein is a biological organ composition wherein the biological organ has been removed from a patient or organ donor and is perfused with a pharmaceutical composition containing the polyoxyethylene/polyoxypropylene block copolymer described below.

The polyoxyethylene/polyoxypropylene copolymer in the pharmaceutical composition administered in the methods described herein has the following chemical formula:

HO(C₂H₄O)ₐ—(C₃H₆O)_{b}—(C₂H₄O)ₐH;

wherein b is an integer such that the hydrophobe represented by (C₃H₆O), or the polyoxypropylene portion, has a molecular weight of approximately 950 to 4000 Daltons, preferably about 1200 to 3500 Daltons, and a is an integer such that the hydrophile portion represented by (C₂H₄O), or the polyoxyethylene portion, constitutes approximately 50% to 95% by weight of the compound. The copolymer has a preferred molecular weight between 5,000 and 15,000 Daltons.

A preferred copolymer is Poloxamer 188 (P188), which has the following chemical formula:

HO(CH₂CH₂O)ₐ—(CH₂CH(CH₃)O)_{b}—(CH₂CH₂O)ₐH;

wherein the molecular weight of the hydrophobe (C₃H₆O), or the polyoxypropylene, is approximately 1750 Daltons and the total molecular weight of the compound is approximately 8400 Daltons.

A further preferred copolymer is purified P188. Purified P188 has reduced low and/or high molecular weight contaminants or substances, wherein the polydispersity value of the polyoxypropylene/polyoxyethylene block copolymer is less than or equal to approximately 1.07, preferably less than or equal to approximately 1.05, or less than or equal to approximately 1.03 as described in U.S. Pat. No. 5,696,298, which is incorporated by reference herein.

### BRIEF DESCRIPTION OF THE DRAWINGS

The FIGURE shows the change in the aggregation index observed for old red blood cells and young red blood cells treated with Poloxamer 188.

### DETAILED DESCRIPTION OF THE INVENTION

### I. Definitions

The terms “a,” “an,” and “the” as used herein includes one or more and include the plural unless the context is inappropriate.

The term “effective amount” as used herein includes an amount of the composition which, when administered to a human or animal, improves blood transfusion and increases tissue oxygenation.

The term “patient” as used herein includes a human or veterinary subject.

The term “blood transfusion” as used herein includes any procedure involving transfused blood cells including apheresis.

The term “jeopardized tissue” as used herein includes tissue having reduced oxygenation or oxygenation below that of a normal individual.

The term “storage lesion” as used herein includes biochemical and biomechanical changes in blood products that result upon storage of the blood products. Storage lesion can adversely affect the viability and function of the blood products in procedures such as transfusion. The adverse biochemical and biomechanical changes include, but are not limited to, lipid oxidation and rearrangement, protein loss, ATP depletion, 2,3-diphosphoglycerate depletion, increased rigidity, release of pro-inflammatory species, comprised deformability, and increased aggregation.

The term “steady state” includes a state of physiological equilibrium especially in connection with a specified metabolic relation or activity. In certain instances, the concept of “steady state” applies to repeated or prolonged administration of dosing regimens. Steady state generally refers to the maintenance of an effective concentration and is less relevant to a bolus dose.

The term “bolus dose” includes achieving an effective concentration for a single point in time, but not necessarily maintaining that dose for longer than 0.5, 1, 2, 3, 4, 5, 6, 7, 8 or 9 hours.

The term “pharmaceutical composition” includes a composition comprising a polyoxyethylene/polyoxypropylene copolymer described herein and optional excipients. In certain instances, the pharmaceutical composition comprises an aqueous injectable solution of the copolymer buffered at a desired pH (about 6) using a buffering agent such as citrate (for example sodium citrate/citric acid) preferably from 0.005 to 0.05M, particularly about 0.01M. In certain instances, pharmaceutical compositions useful in the methods herein are disclosed in WO 94/08596 to The Wellcome Foundation Limited.

### II. Embodiments of the Invention

Methods of enhancing oxygenation of jeopardized tissue are provided herein. The methods are useful for decreasing the need for transfusions, improving the safety and efficacy of blood transfusions, improving organ transplantation, and for the treatment of patients suffering from conditions or disorders that affect the oxygenation of blood and tissues.

For example, the methods described herein are useful for the treatment of several conditions or disorders, including but not limited to: anemia, trauma, hypovolemia, inflammation, sepsis, microvascular compromise, sickle cell disease, acute chest syndrome, peripheral artery disease, myocardial infarction, stroke, peripheral vascular disease, macular degeneration, acute respiratory distress syndrome (ARDS), multiple organ failure, ischemia (including critical limb ischemia), hemorrhagic shock, septic shock, acidosis, hypothermia, and anemic decomposition. The methods described herein are useful for the treatment of patients in need of transfusion, patients undergoing surgery (including plastic surgery), and patients with blood disorders. Furthermore, in one embodiment, the methods described herein are useful for preventing the adverse effects of transfusing a patient with blood or blood products compromised by storage lesion. The compositions and methods described herein are also useful for preserving the function of a donor organ.

In one embodiment of the methods provided herein, an effective amount of a pharmaceutical composition containing the polyoxyethylene/polyoxypropylene copolymer described below is administered to a patient. This method is useful for decreasing the need for blood transfusions or for the treatment of patients suffering from conditions or disorders that affect the oxygenation of blood and tissues.

In accordance with another embodiment, a pharmaceutical composition containing the polyoxyethylene/polyoxypropylene block copolymer described below is combined or admixed with blood or blood products, such as the patient's own blood or the blood of a blood donor and the combination is administered to a patient such as in the form of a blood transfusion. This method is useful for improving the safety and efficacy of blood transfusions.

In accordance with another embodiment, a pharmaceutical composition containing the polyoxyethylene/polyoxypropylene block copolymer described below is administered separately to a patient either prior to, concomitant with, or immediately after a transfusion. This method is useful for improving the safety and efficacy of blood transfusions.

In accordance with another embodiment, a pharmaceutical composition containing the polyoxyethylene/polyoxypropylene block copolymer described below is administered to an organ donor prior to organ donation, an organ to be transplanted into a patient is perfused with the polyoxyethylene/polyoxypropylene block copolymer described below, or the polyoxyethylene/polyoxypropylene block copolymer described below is administered to an organ recipient patient after organ transplantation.

Also provided herein is a biological organ composition, wherein the biological organ has been removed from a patient or organ donor and is perfused with a pharmaceutical composition containing the polyoxyethylene/polyoxypropylene block copolymer described below.

More specifically, methods are provided herein for preventing or reducing tissue ischemia; increasing tissue oxygenation in cases of anemia associated with compromised microvascular function; reversing the effects of storage lesion on RBCs and increasing the ability of RBCs to deliver oxygen to tissues; increasing the safety and effectiveness of transfusing blood with storage lesion; reversing or improving the effects of disorders on the deformability and adhesiveness of RBCs and increasing their ability to deliver oxygen to tissues; increasing the efficacy and safety of blood transfusions for patients with anemia; increasing the efficacy and safety of apheresis; increasing the efficacy and safety of red cell exchange in patients with anemia; increasing the efficacy and safety of blood transfusions of patients undergoing surgery; decreasing the need for blood transfusions during surgery by increasing the ability of RBCs to deliver oxygen; improving cardiac output under conditions where there is decreased deformability of RBCs and decreased ability of RBCs to deliver oxygen to tissues; improving tissue oxygenation during plastic and reconstructive surgery; preventing or reducing multiple organ failure; improving oxygenation of organs prior to and/or during transplantation; preventing or reducing crisis of sickle cell disease; preventing or reducing development of acute chest syndrome of sickle cell disease; preventing or reducing development of ARDS following trauma; improving oxygen delivery to skin flaps in plastic and reconstructive surgery; preventing hypovolemic (hemorrhagic) shock; preventing or reducing septic shock; preventing or reducing development of acute limb syndrome/critical limb ischemia; preventing or reducing deterioration of eyesight in patients with Age Related Macular Degeneration; and preventing or reducing in vivo deterioration of donor organs.

The polyoxyethylene/polyoxypropylene copolymer in the pharmaceutical composition administered in the methods described herein is a linear copolymer having the following chemical formula:

HO(C₂H₄O)ₐ—(C₃H₆O)_{b}—(C₂H₄O)ₐH

wherein b is an integer such that the hydrophobe represented by (C₃H₆O) has a molecular weight of approximately 950 to 4000 Daltons, preferably about 1200 to 3500 Daltons, and a is an integer such that the hydrophile portion represented by (C₂H₄O) constitutes approximately 50% to 95% by weight of the compound.

From the above formula, it will be understood by those of ordinary skill in the art that the value for the integer “a” may differ between the two flanking polyoxyethylene units in a given polymer (in which case the integers for the flanking units can also be considered as “a¹” and “a²” wherein a¹ and a² differ), or may be the same (in which case the integers for the flanking units can also be considered as “a¹” and “a²” wherein a¹ and a² are the same); preferably, the two values for “a” are approximately the same, for example such that the two polyoxyethylene blocks in a given polymer molecule have molecular weights that are approximately equal to one another, for example within about 20% of one another, more preferably within about 10%. It will be understood that the discussions above with respect to “a” on each side of the central hydrophobe block apply equally here and elsewhere in the present application where polymer formulas are provided. The copolymer has a preferred molecular weight between 5,000 and 15,000 Daltons.

The polyoxyethylene/polyoxypropylene copolymer is a surface-active agent, or surfactant, and is formed by ethylene oxide-propylene oxide condensation using standard techniques know to those of ordinary skill in the art. The copolymer is a triblock copolymer of the form poly (ethylene oxide)-poly (propylene oxide)-poly (ethylene oxide).

A preferred copolymer is Poloxamer 188 (P188), CAS No. 9003-11-6, which is a commercially available nonionic tri-block copolymer surfactant composed of a central block of hydrophobic polyoxypropylene flanked by chains of hydrophilic polyoxyethylene. Poloxamer 188 is characterized as a solid, having an average molecular weight of 7680 to 9510 Daltons, a weight percent of oxyethylene of 81.8±1.9%, and an unsaturation level of 0.026±0.008 mEq/g and is represented in the following chemical formula:

HO(CH₂CH₂O)ₐ—(CH₂CH(CH₃)O)_{b}—(CH₂CH₂O)ₐH;

wherein the value of b is such that the molecular weight of the hydrophobe (C₃H₆O) unit is approximately 1750 Daltons and the total molecular weight of the compound is approximately 8400 Daltons. P188 has a molecular weight of approximately 8400 g/mol and a poly (ethylene oxide)-poly (propylene oxide)-poly (ethylene oxide) weight ratio of 4:2:4.

A further preferred copolymer is a purified P188 having reduced low and/or high molecular weight contaminants or substances and a polydispersity less than or equal to approximately 1.07, preferably less than or equal to approximately 1.05, or less than or equal to approximately 1.03. The polydispersity is measured by high performance liquid chromatography (HPLC)-gel permeation chromatography. Purified P188 is described in U.S. Pat. No. 5,696,298.

### P188

Certain polyoxyethylene/polyoxypropylene copolymers have been found to have beneficial biological effects on several disorders when administered to a human or animal. These activities have been described in U.S. Pat. Nos. 4,801,452, 4,837,014, 4,873,083, 4,879,109, 4,897,263, 4,937,070, 4,997,644, 5,017,370, 5,028,599, 5,030,448, 5,032,394, 5,039,520, 5,041,288, 5,047,236, 5,064,643, 5,071,649, 5,078,995, 5,080,894, 5,089,260, RE 36,665 (Reissue of 5,523,492), 5,605,687, 5,696,298 6,359,014, and 6,747,064, and International Applications PCT/US2005/034790, PCT/US2005/037157 and PCT/US2006/006862, and Provisional Patent Application No. 60/995,046, all of which are incorporated herein by reference.

A clinical preparation of P188 can be formulated as a clear, colorless, sterile, non-pyrogenic solution intended for administration with or without dilution. A preferred solution concentration is approximately 15%. In a 15% solution, each 100 mLs contains 15 g of purified P188 (150 mg/ml), 308 mg sodium chloride USP, 238 mg sodium citrate USP, 36.6 mg citric acid USP and water for injection USP Qs to 100 ml. The pH of the solution is approximately 6.0 and has an osmolarity of 312 mOsm/L. A clinical formulation optimally includes bacteriostatic agents or preservatives depending on the intended use.

### Methods of Treatment

The methods of enhancing oxygenation of jeopardized tissue for decreasing the need for transfusions, improving the safety and efficacy of blood transfusions, improving organ transplantation, and for the treatment of patients suffering from conditions or disorders that affect the oxygenation of the blood are accomplished by administering to a patient an effective amount of the pharmaceutically acceptable composition containing the polyoxyethylene/polyoxypropylene copolymer described herein. The effective amount of the composition is administered directly to the patient in accordance with methods well known to those skilled in the art. The pharmaceutical composition is preferably administered by intravenous infusion; however, other routes of administration are contemplated and the preferred route will depend on the disease state and the needs of the patient.

The patient to whom the polyoxyethylene/polyoxypropylene copolymer described herein is administered is a human or non-human having any condition such that there is an inadequate amount of tissue oxygenation.

The effective amount is preferably delivered by administration as an infusion such as a single bolus infusion or a continuous infusion administered either once or multiple times. The effective amount will preferably target a concentration in the circulation of the patient of between approximately 0.05 mg/ml and 10 mg/ml depending upon the duration of the infusion and the needs of individual patients. In a preferred embodiment for intermittent bolus infusions at weekly, two week or three week intervals, the target range is between approximately 0.5 to 5.0 mg/ml. In a preferred embodiment for continuous infusions, the target range is approximately 0.1 to 1 mg/ml, preferably approximately 0.5 mg/ml. These ranges are not intended to be limiting and will vary based on the needs and response of the individual patient. The amount of the dose of polyoxyethylene/polyoxypropylene copolymer sufficient to achieve the target concentration is readily determined by one of ordinary skill in the art following routine procedures. The pharmaceutical composition is typically administered at a concentration of between approximately 0.5% to 15%. The composition may also be delivered in a more dilute or more highly concentrated dosage depending on the needs of the individual patient. The actual amount or dose of the composition required to elicit the desired effect will vary for each individual patient depending on the response of the individual. Consequently, the specific amount administered to an individual will be determined by routine experimentation and based upon the training and experience of one skilled in the art.

The effective amount of polyoxyethylene/polyoxypropylene copolymer will depend on the degree of tissue ischemia, the disease state or condition and other clinical factors including, but not limited to, such factors as the patient's weight and kidney function as is known in the art. The methods described herein contemplate a single continuous infusion, multiple continuous infusions, or bolus administrations administered once or multiple times over an extended period of time for as long as needed to achieve the desired effect.

With regard to improving the safety and efficacy of blood transfusions, improvement in tissue oxygenation before, during or after transfusion is accomplished by administering to a patient an effective amount of the pharmaceutically acceptable composition containing the polyoxyethylene/polyoxypropylene copolymer, as described herein. The effective amount of the composition is administered directly to the patient, admixed with the blood to be transfused, or administered as various combinations thereof. As mentioned above, the preferred copolymer is P188 provided as a substantially purified composition, preferably in a pharmaceutically acceptable formulation. The formulation is typically administered by intravenous infusion; however, other routes are contemplated and the preferred route will depend on the disease state and the needs of the patient.

The effective amount of the polyoxyethylene/polyoxypropylene copolymer is delivered by admixing the pharmaceutical composition directly with the blood to be transfused or administered as a separate infusion immediately prior to transfusion, concomitant with transfusion, or immediately following transfusion or as combinations thereof. When administered as a separate infusion the effective amount may be administered as a single bolus administration administered either once or multiple times, or a continuous infusion administered either once or multiple times. Whether admixed with the blood to be transfused or administered separately, the effective amount will preferably target a concentration in the circulation of the transfused patient of between 0.05 mg/ml and 10.0 mg/ml; however, this range is not intended to be limiting and will vary based on the needs and response of the individual patient. The target concentration in the circulation is generally maintained for up to 72 hours following transfusion; however, this time is not meant to be limiting. The amount of the pharmaceutically acceptable copolymer composition admixed with transfused blood or the dose to achieve the target concentration is readily determined by one of ordinary skill in the art following routine procedures. The pharmaceutically acceptable copolymer composition is typically admixed with the blood to be transfused or administered separately at a concentration of between 0.5% to 15%. The composition may also be delivered in a more dilute or more highly concentrated dosage. When administered separately the preferred route of administration is intravenous infusion, although other routes may also be used. The actual amount or dose of the composition required to elicit the desired effect will vary for each individual patient depending on the response of the individual. Consequently, the specific amount administered to an individual will be determined by routine experimentation and based upon the training and experience of one skilled in the art.

The effective amount of the polyoxyethylene/polyoxypropylene copolymer will depend on the amount of blood transfused, the degree of tissue ischemia, the disease state or condition and other clinical factors including, but not limited to, such factors as the patient's weight and kidney function as is known in the art. The methods described herein contemplate a single continuous infusion, multiple continuous infusions, or bolus administrations administered once or multiple times over an extended period of time for as long as needed to achieve the desired effect.

It is to be understood that the methods provided herein have applications for both human and veterinary use.

The pharmaceutical compositions provided herein are suitable for various routes of administration including, but not limited to: subcutaneous, intraperitoneal, intramuscular, intrapulmonary, and intravenous. The formulations may be presented in a unit or multi-dose form and may be prepared by conventional pharmaceutical techniques. Such techniques include the step of bringing into association the active ingredient and the pharmaceutical carrier(s) or excipient(s).

Formulations suitable for parenteral administration include aqueous and non-aqueous sterile injection solutions, which optimally contain anti-oxidants, buffers, bacteriostats and solutes that render the formulation compatible with the intended route of administration. The formulations may be presented in unit-dose or multi-dose containers, for example, sealed ampoules and vials, prefilled syringes or other delivery devices and may be stored in an aqueous solution, dried or freeze-dried (lyophilized) condition, requiring only the addition of the sterile liquid carrier, for example, water for injections, immediately prior to use.

The method provided herein is further illustrated by reference to the following examples, which are not to be construed in any way as imposing limitations upon the scope thereof. On the contrary, it is to be clearly understood that other embodiments, modifications, and equivalents thereof which, after reading the description herein, may suggest themselves to those skilled in the art without departing from the spirit of the present invention and/or the scope of the appended claims.

### Methods for Remedying Storage Lesion

After removal from the body and with the added effect of storage, RBCs undergo biochemical and biomechanical changes (many irreversible) that adversely affect their viability and function. These adverse changes include oxidation and rearrangement of lipids, loss of proteins, and depletion of ATP and 2,3-diphosphoglycerate. In storage, RBCs continuously acquire defects in their membrane through shedding vesicles and other processes contributing to increased rigidity. Moreover, during storage, bioactive by-products and ions (hemoglobin, lipids, and potassium), some with pro-inflammatory effects, are released from RBCs and accumulate in the stored blood units where they can cause adverse reactions in a recipient. Red cell deformability and aggregation have also been shown to be significantly affected after storage. These parameters hinder the ability of red cells to traverse the microvasculature resulting in decreased local oxygen delivery (Kiraly, L. N., S. Underwood, J. A. Differding, and M. A. Schreiber. 2009. Transfusion of aged packed red blood cells results in decreased tissue oxygenation in critically injured trauma patients. J Trauma 67:29-32). These changes are collectively called storage lesion.

Transfusion of blood that is stored for prolonged periods (but still within the currently accepted maximum allowed storage time of 42 days) has been linked to increased risk of complications and reduced survival in patients undergoing cardiac surgery and in other patient populations (O'Keeffe, S. D., D. L. Davenport, D. J. Minion, E. E. Sorial, E. D. Endean, and E. S. Xenos. Blood transfusion is associated with increased morbidity and mortality after lower extremity revascularization. J Vasc Surg 51:616-621). Endogenous RBC deformability is thought to be a critical factor in micro-vascular blood flow. RBC transfusions improved RBC deformability in patients with sepsis, probably by replacing rigidified RBCs by more functional, or less dysfunctional, exogenous RBCs. Hence, transfusions may be deleterious when performed in patients where storage has impaired RBC deformability. This may explain why RBC transfusion may decrease sublingual microcirculation when it is essentially normal at baseline but improve it when it is decreased at baseline (Sakr, Y., M. Chierego, M. Piagnerelli, C. Verdant, M. J. Dubois, M. Koch, J. Creteur, A. Gullo, J. L. Vincent, and D. De Backer. 2007. Microvascular response to red blood cell transfusion in patients with severe sepsis. Crit Care Med 35:1639-1644).

As described above, the methods described herein are useful for preventing or reducing the adverse effects of transfusing a patient with blood or blood products compromised by storage lesion. In particular, the safety and effectiveness of transfusing blood with storage lesion can be increased using the methods of the present invention.

Accordingly, one embodiment of the present invention provide a method for preventing or reducing the adverse effects of transfusing a patient with blood or a blood product compromised by storage lesion. The method includes administering to a patient a pharmaceutical composition comprising an effective amount of a polyoxyethylene/polyoxypropylene copolymer having chemical formula HO(C₂H₄O)ₐ—(C₃H₆O)_{b}—(C₂H₄O)ₐH, wherein b is an integer such that the hydrophobe represented by (C₃H₆O) has a molecular weight of approximately 950 to 4000, preferably approximately 1200 to 3500, and a is an integer such that the hydrophile portion represented by (C₂H₄O) constitutes approximately 50% to 95% by weight of the compound, and a pharmaceutically acceptable carrier.

In certain instances, b is an integer of from about 15 to about 70, such as from about 15 to about 60, or from about 15 to about 30, or any of the numbers in between. In some instances, b is about 15, 16, 17, 18, 19, 20, 21, 22, 23, 24, 25, 26, 27, 28, 29, or 30. In certain aspects, the integers for the flanking units with the subscript “a” can be considered as “a¹” and “a²” wherein a¹ and a² can differ or are the same values. In some instances, a is an integer of about 45 to about 910, such as 90, 100, 200, 300, 400, 500, 600, 700, 800, or 900. In some other instances, a is an integer from about 10 to about 215, such as 10, 20, 30, 40, 50, 60, 70, 80, 100, 125, 150, 175, 200 or 215. In still other instances, a is about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70. A skilled artisan will appreciate that these values are average values. That is, the values for a and b represent an average, as in a preferred aspect, the polymeric molecules are a distribution or population of molecules and therefore the actual values of a and b within the population will constitute a range of values.

In some embodiments, the polyoxyethylene/polyoxypropylene copolymer has the formula: HO(CH₂CH₂O)ₐ—(CH₂CH(CH₃)O)_{b}—(CH₂CH₂O)ₐH, wherein the molecular weight of the hydrophobe (CH(CH₃)CH₂O) is approximately 1750 Daltons and the total molecular weight of the compound is approximately 8400 Daltons. In some embodiments, the polyoxyethylene/polyoxypropylene copolymer is purified to reduce low and/or high molecular weight contaminants or substances so that the polydispersity value is less than or equal to approximately 1.07.

The blood or blood product can be derived from any suitable source. In general, the blood or blood product for transfusion in a human patient is obtained from a human donor. In some embodiments, the blood or the blood product is non-autologous blood or a non-autologous blood product; i.e., the donor is other than the patient. In some embodiments, the blood or blood product is collected from the patient and administered to the same patient during transfusion.

In some embodiments, the pharmaceutical composition is admixed with the blood or the blood product to be transfused to form a blood admixture. prior to transfusion. In some embodiments, the pharmaceutical composition is substantially free of the blood or the blood product prior to transfusion. The pharmaceutical composition can be admixed with the blood or the blood product at any time after it is collected from a donor or other source. The blood or blood product can be stored before it is mixed with the copolymer composition. The blood or blood product can be stored, for example, for 30 minutes, 1 hour, 2 hours, 4 hours, 6 hours, 8 hours, 12 hours, 16 hours, one day, two days, three days, four days, five days, six days, one week, two weeks, three weeks, four weeks, five weeks, six weeks, or longer periods before it is mixed with the copolymer composition. In some embodiments, the blood or blood product is stored for at least two weeks before it is mixed with the copolymer composition. Alternatively, the blood or blood product can be mixed with the copolymer composition to form a blood admixture. The admixture can be stored, for example, for 30 minutes, 1 hour, 2 hours, 4 hours, 6 hours, 8 hours, 12 hours, 16 hours, one day, two days, three days, four days, five days, six days, one week, two weeks, three weeks, four weeks, five weeks, six weeks, or longer periods before it is used for transfusion. In some embodiments, the admixture is stored for at least two weeks before it is used for transfusion. One of skill in the art will appreciate that the storage period will depend in part on the specific blood product and the storage conditions. In some embodiments, the pharmaceutical composition is administered to the patient prior to, concomitant with, or immediately after transfusion with the blood or the blood product.

In some embodiments, the blood or the blood product comprises one or more components selected from white blood cells, red blood cells, and platelets. In some embodiments, the blood or the blood product comprises red blood cells.

In some embodiments, the method increases the ability of the red blood cells to deliver oxygen to a tissue in the patient. Tissue can be jeopardized due to a number of conditions, including any of those described herein. In some embodiments, the tissue is jeopardized by anemia, trauma, hypovolemia, inflammation, sepsis, or microvascular compromise. In some embodiments, compromised deformability of red blood cells is reversed or improved. In some embodiments, red blood cell adhesiveness is reduced or prevented. In some embodiments, red blood cell aggregation is reduced or prevented.

Viability of the blood or the blood products can be assessed by a number of criteria. Blood cell morphology and rheology can be analyzed to determine the suitability of the blood or blood product for transfusion. The analysis can be made with and without addition of the copolymer composition to assess improvement of the blood or blood product upon mixing with the composition. Alternatively, ATP or 2,3-diphosphoglycerate in the cells can be quantified using known procedures to determine the viability of the blood or blood product. Other characteristics of the blood or blood product can be used to assess quality prior to transfusion.

Any suitable amount of copolymer can be used in the compositions and methods of the invention. In general, the pharmaceutical composition will contain from about 0.005% to about 25% of the copolymer by weight. The pharmaceutical composition can contain, for example, 0.005%, or 0.025%, or 0.05%, or 0.1%, or 0.25%, or 0.5%, or 1%, or 2.5%, or 5%, or 10%, or 12.5%, or 15%, or 20%, or 25% or more of the copolymer by weight. In some embodiments, the composition comprises the copolymer in an amount of from about 0.5% to about 20% such as 0.5, 1, 2, 3, 4, 5, 6, 7, 8, 9, 10, 11, 12, 13, 14, 15, 16, 17, 18, 19, or 20% by weight. As described above, blood admixtures containing blood or blood product and the copolymer composition can be stored and used for transfusion. The blood admixture can contain any suitable amount of copolymer. In some embodiments, the blood admixture includes the copolymer in an amount of from about 0.05 mg/mL to about 5 mg/mL. In some other embodiments, the blood admixture includes the copolymer in an amount of about 0.5 mg/mL.

In some embodiments, administering the pharmaceutical compositions results in a concentration of the copolymer in the circulation of the patient of from about 0.01 mg/mL to about 10 mg/mL. In some embodiments, administering the pharmaceutical composition results in a concentration of the copolymer in the circulation of the patient of about 0.01 mg/mL to about 1 mg/mL such as 0.01, 0.02, 0.03, 0.04, 0.05, 0.06, 0.07, 0.08 0.09, 0.1, 0.2, 0.3, 0.4, 0.5, 0.6, 0.7, 0.8, 0.9, or 1.0 mg/mL. In one preferred embodiment, the concentration is about 0.3 to about 0.7 mg/mL such as 0.5 mg/mL. In some embodiments, the concentration in the circulation is targeted for up to 72 hours following transfusion. In order to achieve a desired concentration of the copolymer in the circulation of the patient, additional doses of a copolymer composition can be administered. Accordingly, some embodiments of the invention provide methods as described above, further including administering to the patient an additional amount of the pharmaceutical composition, wherein the additional amount is sufficient to result in a concentration of the copolymer in the circulation of the patient of from about 0.05 mg/mL to about 10 mg/mL. In some embodiments, the additional amount of the pharmaceutical composition is sufficient to result in a concentration of the copolymer in the circulation of the patient of about 0.5 mg/mL. In one embodiment, a stored blood unit has pharmaceutical composition contained therein. After transfusion, a second administration to the patient of the pharmaceutical composition, which is sufficient to result in a concentration of the copolymer of from about 0.05 mg/mL to about 10 mg/mL.

The compositions can be administered to a patient via any suitable route according to the methods of the invention. In some embodiments, the composition is administered via intravenous infusion. In some embodiments, the formulation is administered as a single continuous infusion, multiple continuous infusions, a single bolus administration, or multiple bolus administrations.

### III. Examples

### Example 1

### Patient with Trauma Needing Transfusion

A 42-year-old man is admitted to the trauma intensive care unit following a motor vehicle accident. The next day he is relatively stable with blood pressure of 130/65 and had no evidence of sepsis. However, when his hematocrit falls to 22%, a transfusion of a unit of packed red blood cells is ordered. A near infrared tissue spectrometer is used to record tissue oxygen saturation values (StO₂). The spectrometer is placed on the thenar eminence. Tissue oxygenation measurements are made continuously and recorded every three minutes. Data collection starts one hour before the start of transfusion and ends six hours after the transfusion was complete.

Baseline StO₂ values before the transfusion fluctuate between 86% and 87%. The transfusion is accomplished with packed red blood cells that are 39 days old. The patient's blood pressure and heart rate do not change significantly. However, the StO₂ declines to a value of 81% at 2 hours after starting the transfusion. At that point the patient is infused with 200 mg/kg of P188 over a period of ten minutes. The StO₂ values then rise to 91% and persist at that level through the end of the study. There are no significant changes in blood pressure or heart rate.

### Example 2

### Patient with Trauma Needing Transfusion

A critically ill trauma patient is transfused with one unit of packed RBC, which increases mean hemoglobin from 9.2 g/dl to 10.1 g/dl. However, there are no changes in oxygen delivery (490 ml/min/m²), oxygen consumption (210 ml/min/m²), or mixed venous PO/(37 Torr). One hour after the transfusion, the patient is infused with P188 (200 mg/kg) over a period of 10 minutes. Within the next hour, oxygen delivery increases to 600 ml/min/m²), oxygen consumption increases to 300 ml/min/m², and mixed venous PO increases to 60 Torr.

### Example 3

### Patient with Sickle Cell Prodrome

A 10-year-old girl is brought to the hospital because of a prodrome of impending acute crisis of sickle cell disease. Prior experience indicated that such prodromes are typically followed by acute crisis. She is infused with P188 (100 mg/kg) over ten minutes followed by a continuous infusion of 30 mg/kg/hour for six hours. The prodrome resolves, and the crisis does not develop.

### Example 4

### Patient with Sickle Cell to Prevent Acute Chest Syndrome (ACS)

A 12-year-old girl, hospitalized with an ongoing sickle cell painful crisis, develops a new pulmonary infiltrate and shows worsening of vital signs. StO₂ measurements fall from 70% to 50%. Her arterial oxygen saturation is 76% despite aggressive respiratory support. The patient is treated with apheresis exchange transfusion targeting 1.5 red cell volumes. An infusion of P188 at 200 mg/kg/hour is started 15 minutes prior to transfusion. The P188 is diluted with normal saline to a concentration to deliver the desired dosage while maintaining proper hydration with the aid of a programmable infusion pump. Within one hour of starting treatment the patients O₂ saturation is over 90%, StO₂ has increased to 75% and vital signs have improved. The P188 infusion is continued for 12 hours, the patient continues to improve, and there is no evidence of hyperviscosity or other transfusion related complications.

### Example 5

### Patient with Severe Anemia Refuses Transfusion

A 67-year-old man loses seven units (3500 ml) of blood during a surgical operation but refuses blood transfusion on religious grounds. On arrival to the ICU he has a hemoglobin of 7.9 μm, is tachycardiac (150-160 beats/min), tachypneic (32-35 breaths/min), diaphoretic and lethargic. Blood pressure is normal at 130-150/70-90 mm Hg. Arterial oxygen saturation is 95% while breathing oxygen at 3 L/min by nasal cannula. He is infused with a colloid (2 units of hetastarch) and crystalloid fluids at 150 mL/hr. The next morning, his hemoglobin falls to a dangerous level, 3.0 g/dl, due to fluid equilibrium (because there was no active bleeding). A pulmonary artery catheter is inserted for better monitoring of his condition and he is given 100% oxygen to breathe. Mixed venous oxygen saturation (SvO₂) falls to 50% (normal=60%-80%) and TcPO₂ is 60.

P188 (200 mg/kg) is administered over 15 minutes followed by a continuous infusion of 30 mg/kg/hour) for 24 hours. The SvO₂ rises to 75% within an hour and TcPO₂ rises to 80 ameliorating the dangerous condition. Subsequently, P188 is administered at 30 mg/kg/hour when the SvO2 falls below 60%. The patient is also given erythropoietin, folic acid and intravenous iron to stimulate red cell production. His hemoglobin gradually increases, and he is discharged from the ICU in ten days and from the hospital eight days later.

### Example 6

### Patient with Gastrointestinal Bleeding Refuses Transfusion

A 49-year-old man suffers gastrointestinal (GI) bleeding that is controlled with conventional therapy. However, his hemoglobin falls to 4.7 g/dl (hematocrit 14%). He refuses transfusion on religious grounds. Pulmonary and radial artery catheters are placed to monitor vital functions. Administration of oxygen by mask increases arterial partial pressure of oxygen (80 mmHg to 350 mmHg), blood oxygen content (5.2 volume % to 6.5 volume %) and mixed venous oxygen content (51 mmHg to 80 mmHg). However, oxygen alone fails to increase oxygen consumption (190 ml/min to 189 ml/min). The patient is infused with P188 (500 mg/kg) over a period of two hours. His oxygen consumption immediately after the infusion rises to 255 ml/min while his blood oxygen content and cardiac output change very little. He recovers fully.

### Example 7

### Patient Undergoing Plastic Surgery

A 48-year old patient undergoes breast reconstruction surgery. Continuous 72 hour NIRS monitoring of postoperative skin flaps produced by plastic-reconstructive surgery can detect tissue hypoxia. A breast flap is monitored continuously with StO₂ after surgery. The value stabilizes at 30%, a value too low for optimal healing. The patient is infused with P188 (100 mg/kg over 15 minutes followed by a continuous infusion of 30 mg/kg/hour for 48 hours. The StO2 rises to 60% and the flap heals uneventfully.

### Example 8

### Patient with PAD Develops Pain at Rest

A 59-year old patient with Peripheral Artery Disease (PAD) is checked in to the hospital reporting pain. His TcPO₂ is measured and is found to be too low, resulting in inadequate oxygenation of leg tissue. The patient's StO₂ in his legs is also measured and is found to be too low. The patient is then infused with P188 (200 mg/kg). As a result, the TcPO₂ is improved and the patient's pain ceases. Amputation of the legs is not necessary.

### Example 9

### Patient with Sepsis Develops Falling StO₂

A 72-year-old woman is diagnosed with sepsis syndrome by standard criteria. Tissue oxygenation measured by StO₂ declines to 60%. Hemodynamic profiles with serum lactate levels are obtained before and after packed red blood cells are given. Oxygen uptake fails to increase with transfusion, corresponding to increased arterial and mixed venous oxygen content. She is then infused with P188 (200 mg/kg). Her oxygen uptake and StO₂ both increase.

### Example 10

### Patient is Fatally Injured; Need to Preserve Organ Function for Donation

A 32-year-old man receives a fatal head injury in a motorcycle accident. After declaration of brain death, his family agrees to donate his organs for transplantation. He is in shock and maintained on a ventilator. P188 (500 mg/kg) is infused intravenously to prevent ischemic damage to the kidneys and other organs before they are removed for transplant.

### Example 11

### Normal Patient

A normal 26-year-old woman is infused with 400 mg/kg of P188. There are no changes in blood any vital signs, oxygen consumption, TcpO₂ or StO₂.

### Example 12

### Effect of Poloxamer 188 on the Aggregation of Older and Younger Red Blood Cells

It is generally accepted that under normal blood flow conditions, circulating red blood cells (RBC's) are not aggregated and flow as individual cells. Under certain pathological conditions (such as during an acute inflammatory process) RBC's can aggregate into masses of cells. In the medical literature the presence of these masses of aggregated RBC's in the circulation has been termed sludged blood. Sludged blood results in impaired tissue perfusion, and tissue ischemia. Accordingly, changes resulting in an increase in RBC aggregation have an inverse correlation to blood flow.

The lifespan of a RBC in the circulation is about 120 days. As cells age they become less deformable and otherwise dysfunctional. It is generally believed that the RBC in blood stored for transfusion continues to age or even experiences accelerated aging even though it is stored with preservatives. As discussed above, increased RBC aggregation is a reasonable measure of the age-related dysfunction of RBC's. It has been shown that older RBC show a markedly increased aggregation index compared to younger RBC.

Surprisingly, Poloxamer 188 was found to restore functionality in older RBC's, rendering them more like younger RBC's i.e., rejuvenate the older cells. This phenomenon was examined by comparing the effect of Poloxamer 188 on the aggregation in older and younger RBC's.

### Methods

Blood was obtained from 5 healthy adult donors. The RBC were age separated by high speed centrifugation (younger RBC's are more dense than older RBC's) and re-suspended to a final hematocrit of about 40% in 3% dextran 70 containing 0, 1.0, or 5.0 mg/ml poloxamer 188. Aggregations comparing the older RBC's with younger RBC's were carried out using a computerized Myrenne aggregometer. The system measures increases in light transmission due to the formation of the RBC aggregates.

### Results

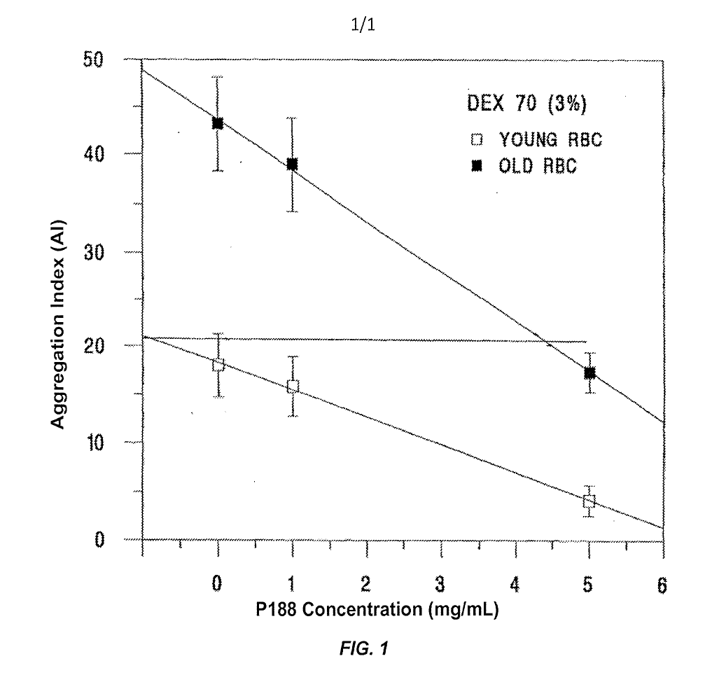

Older RBC's were observed to aggregate more than young RBC's. Under the test conditions, a two-fold increase in aggregation index was observed for older vs. younger RBC's in the absence of poloxamer 188 (zero concentration). The aggregation of both old RBC's and young RBC's was reduced by poloxamer 188 in a concentration related manned. The effect on older RBC's, however, was greater. See, FIG. 1. The slope of the dose response curve observed for older RBC's was more than twice that observed for younger RBC's. Of particular interest was the observation that at the concentration of 5.0 mg/ml of poloxamer, the extent of aggregation of older RBC's was similar to that of the younger RBC's without poloxamer. This suggests that poloxamer 188 can improve the age-related aggregation dysfunction of older RBC's in a way that renders them more like younger RBC with regard to their tendency to aggregate.

### Example 13

### Prevention or Reduction of Storage Lesion in Whole Blood

450 mL of whole blood is collected from an adult male donor via the median cubital vein. The blood is mixed with 63 mL of a citrate-phosphate-dextrose buffer (3% sodium citrate, 3% anhydrous dextrose, 0.3% citric acid monohydrate; 0.25% sodium phosphate) containing 0.05% P188 by weight (0.5 mg/mL). The blood is stored for 48 hours at 20° C. A sample without P188 is reserved for comparative analysis. After the storage period, 2,3-diphosphoglycerate (2,3-DPG) is quantified using a phosphoglycerate mutase assay (Roche Applied Science) according to the manufacturer's instruction. 2,3-DPG is lower in blood stored without the P188 than in the blood stored with the P188.

### Example 14

### Comparison of Blood Flow Velocity with Transfusion with and without P188

An 8 year old boy with sickle cell disease that is on a chronic transfusion program undergoes evaluation of his microcirculatory function by computer assisted video microscopy (CAIM) of the bulbar conjunctiva one hour prior to his scheduled exchange transfusion. The study reveals a flow velocity of 1.2 mm/sec. The CAIM results are slightly lower than his typical steady-state (non-crisis) values which generally range between 1.4-1.6 mm/sec. He undergoes the scheduled exchange which involves withdrawal of 10 mL/Kg of blood by phlebotomy and immediate infusion of 15 mL/Kg of white blood cell reduced red blood cells (RBC's) matched for E, C and Kell antigens.

One hour following exchange transfusion, a repeat CAIM shows the flow velocity has decreased by 0.5 mm/sec to 0.7 mm/sec. His blood pressure remains unchanged from the pre-transfusion value. At his next scheduled transfusion the boy undergoes a similar CAIM of the bulbar conjunctiva one hour prior to his scheduled transfusion where he is observed to have a flow velocity of 1.1 mm/sec. He is treated with a bolus infusion of P188 at 100 mg/kg immediately prior to transfusion and undergoes the scheduled exchange which involves withdrawal of 10 mL/Kg of blood by phlebotomy and immediate infusion of 15 mL/Kg of white blood cell reduced red blood cells (RBC's) matched for E, C and Kell antigens. One hour following exchange transfusion, a repeat CAIM shows that the flow velocity has improved by 0.4 mm/sec to a rate of 1.5 mm/sec. His blood pressure is unchanged from the pre-transfusion value.

All references cited herein are hereby incorporated by reference. Modifications and variations of the present methods will be obvious to those skilled in the art from the foregoing detailed description. Such modifications and variations are intended to fall within the scope of the appended claims.

What is claimed is:

1. A composition, comprising:
blood or a blood product to be transfused; and
a polyoxyethylene/polyoxypropylene copolymer having the chemical formula:
HO(C₂H₄O)ₐ—(C₃H₆O)_{b}—(C₂H₄O)ₐH,
wherein:
the blood or blood product is compromised by storage lesion;
b is an integer such that the hydrophobe represented by (C₃H₆O) has a molecular weight of approximately 950 daltons to 4000 daltons; and
each a, which can be the same or different, is an integer such that the hydrophile portion represented by (C₂H₄O) constitutes approximately 50% to 90% by weight of the copolymer.

2. The composition of claim 1, wherein the composition comprises the copolymer in an amount of from about 0.5% to about 15% by weight.

3. The composition of claim 1, wherein b is an integer such that the hydrophobe represented by (C₃H₆O) has a molecular weight of approximately 1200 daltons to 3500 daltons.

4. The composition of claim 1, wherein:
the polyoxyethylene/polyoxypropylene copolymer has the following chemical formula: 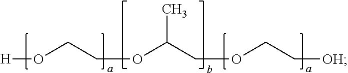
the molecular weight of the hydrophobe (C₃H₆O) is approximately 1750 daltons; and
the total molecular weight of the copolymer is approximately 8400 daltons.

5. The composition of claim 4, wherein the composition comprises the copolymer in an amount of from about 0.5% to about 15% by weight.

6. The composition of claim 1, wherein the polydispersity value of the copolymer is less than or equal to approximately 1.07.

7. The composition of claim 4, wherein the polydispersity value of the copolymer is less than or equal to approximately 1.07.

8. The composition of claim 1, wherein compromised deformability of the red blood cells in the composition is reversed or improved.

9. The composition of claim 1, wherein adhesiveness and/or aggregation of red blood cells in the composition is reduced or prevented.

10. The composition of claim 7, wherein the composition comprises the copolymer in an amount of from about 0.5% to about 15% by weight.

11. The composition of claim 1, wherein the composition is formulated for administration via intravenous infusion.

12. The composition of claim 1, wherein the composition is formulated for intravenous administration as a single continuous infusion, multiple continuous infusions, a single bolus administration or multiple bolus administrations.

13. A method of improving blood for transfusion, comprising mixing the blood or blood product to be transfused with a polyoxyethylene/polyoxypropylene copolymer to produce a composition of claim 1, wherein:
the blood or blood product is compromised by storage lesion;
the polyoxyethylene/polyoxypropylene copolymer has the chemical formula:
HO(C₂H₄O)ₐ—(C₃H₆O)_{b}—(C₂H₄O)ₐH;
b is an integer such that the hydrophobe represented by (C₃H₆O) has a molecular weight of approximately 950 daltons to 4000 daltons; and
a is an integer such that the hydrophile portion represented by (C₂H₄O) constitutes approximately 50% to 90% by weight of the compound.

14. The method of claim 13, wherein b is an integer such that the hydrophobe represented by (C₃H₆O) has a molecular weight of approximately 1200 daltons to 3500 daltons.

15. The method of claim 13, wherein the copolymer is a Poloxamer 188

16. The method of claim 13, wherein the polyoxyethylene/polyoxypropylene copolymer has a polydispersity value less than or equal to approximately 1.07.

17. The method of claim 13, wherein the composition comprises the copolymer in an amount of from about 0.5% to about 15% by weight.

18. The method of claim 15, wherein the composition comprises the copolymer in an amount of from about 0.5% to about 15% by weight.

19. The method of claim 18, wherein the polyoxyethylene/polyoxypropylene copolymer has a polydispersity value less than or equal to approximately 1.07.

20. A combination, comprising:
a) a first composition containing a polyoxyethylene/polyoxypropylene copolymer that has the chemical formula:
HO(C₂H₄O)ₐ—(C₃H₆O)_{b}—(C₂H₄O)ₐH, wherein:
b is an integer such that the hydrophobe represented by (C₃H₆O) has a molecular weight of approximately 950 daltons to 4000 daltons; and
a is an integer such that the hydrophile portion represented by (C₂H₄O) constitutes approximately 50% to 90% by weight of the compound; and
b) a second composition that is blood or a blood product for transfusion, wherein the blood or blood product is compromised by storage lesion.

21. The combination of claim 20, wherein b is an integer such that the hydrophobe represented by (C₃H₆O) has a molecular weight of approximately 1200 daltons to 3500 daltons.

22. The combination of claim 20, wherein the copolymer has the formula: 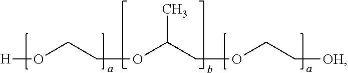
wherein the molecular weight of the hydrophobe (C₃H₆O) is approximately 1750 daltons, and the total molecular weight of the compound is approximately 8400 daltons.

23. The combination of claim 20, wherein the polyoxyethylene/polyoxypropylene copolymer has a polydispersity value less than or equal to approximately 1.07.
